(19) United States
(12) Patent Application Publication
(10) Pub. No.: US 2015/0037826 A1
(43) Pub. Date: Feb. 5, 2015

(54) STANDARD FOR QUANTIFYING PATHOGENIC AGGREGATES FROM PROTEINS PRODUCED NATURALLY IN THE BODY

(71) Applicant: FORSCHUNGSZENTRUM JUELICH GMBH, Juelich (DE)

(72) Inventors: Dieter Willbold, Juelich (DE); Susanne Aileen Funke, Sonnefeld (DE)

(21) Appl. No.: 14/366,941

(22) PCT Filed: Dec. 21, 2012
(86) PCT No.: PCT/EP2012/076551

§ 371 Date: Jun. 19, 2014

(30) Foreign Application Priority Data
Dec. 23, 2011 (DE) 102011057019.5

(51) Int. Cl. G01N 33/68; C07K 14/47

(52) U.S. Cl. CPC G01N 33/6896; C07K 14/4711; G01N 2333/4709; G01N 2800/2835; G01N 2800/2828; G01N 2800/2821
USPC 435/7.94; 530/350; 530/403; 435/69.3; 436/501; 435/7.92

(57) ABSTRACT

The invention relates to standards for quantifying pathogenic aggregates or oligomers of endogenous proteins which characterize a protein aggregation disease, amyloid degeneration or protein misfolding diseases and use of these standards for quantifying these pathogenic aggregates or oligomers.

The invention relates to standards for quantifying pathogenic aggregates or oligomers of endogenous proteins which characterize a protein aggregation disease, amyloid degeneration or protein misfolding diseases and use of these standards for quantifying these pathogenic aggregates or oligomers.

A heterogeneous group of clinical conditions the common criterion whereof is in many cases (but not exclusively) extracellular, systemic or local deposition of a specific protein in each case in the ordered conformation of beta sheet structure is described as protein misfolding diseases or protein aggregation diseases or amyloid degeneration. This group also includes Alzheimer's disease (AD, Alzheimer's dementia, Latin=Morbus Alzheimer) or Parkinson's disease. In modern society, age-related dementia is an ever greater problem since owing to the increased life expectation ever more people are affected by it and the disease thus has repercussions on the social insurance systems and their financial viability.

Pathological aggregates of endogenous proteins, such as for example oligomers or fibrils, occur in many neurodegenerative diseases. In Alzheimer's dementia, for example, amyloid-beta peptide deposits (A-beta peptide deposits) are found in the brain and in Parkinson's disease synuclein deposits. The amyloid-beta peptide deposits (or plaques, consisting of peptide fibrils) are however merely the final stage of a process which begins with the cleavage of monomeric amyloid-beta peptides from APP (amyloid precursor protein), then forms neurotoxic amyloid-beta peptide oligomers and finally ends with the deposition of amyloid-beta peptide fibrils in plaques. The main pathological feature of AD is the formation of senile or amyloid plaques, consisting of the A-beta peptide. Furthermore, neurofibrillar deposits of the tau protein are formed. The precursor protein of the A-beta peptide, APP, is located in the cell wall of the neurones. Through proteolytic degradation and possibly subsequent modification, A-beta fragments of various length and nature, such as for example A-beta 1-40, A-beta 11-40, A-beta 1-42, A-beta 11-42 or pyroGluA-beta 3-42 and pyroGluA-beta 3-40 are formed from this. Monomeric A-beta peptides are also formed in the healthy body throughout life.

According to the amyloid cascade hypothesis from the 1990's, the A-beta deposits in the form of plaques are the triggers of the disease symptoms. In recent years, however, various studies are indicating that in particular the small, freely diffusing A-beta oligomers possess the greatest toxicity and are responsible for the onset and progression of AD. Thus aggregates of the A-beta peptides are directly linked with AD pathogenesis.

At present, however, a reliable diagnosis of AD is only possible after the appearance of prominent clinical symptoms, and a reliability of at most 90% is assumed in this. The only previously certain diagnostic possibility at present exists only after the patient's death, through histological evidence of various changes in the brain.

Accordingly there is a need for methods for the identification and quantitative estimation of pathological aggregates or oligomers of endogenous proteins which cause and/or characterize a protein aggregation disease, amyloid degeneration or a protein misfolding disease.

Only a few methods for the characterization and quantification of pathogenic aggregates or oligomers of endogenous proteins which cause a protein aggregation disease, amyloid degeneration or a protein misfolding disease in tissues and body fluids have so far been described.

For the development of such methods, and in order to ensure the comparability of the results determined therewith, precisely defined standards, i.e. precisely characterized (synthetic) polymers are necessary. For use as standards these must be available in various sizes and forms, which must however be precisely defined.

However, the aggregation of peptides is determined by a multitude of factors, such as for example temperature, salt content of the sample, company manufacturing the proteins, purity, etc. As a result, the production of polymers as standards by aggregation of monomer peptides for test development and validation is difficult to reproduce.

Moreover, for example prepared A-beta oligomers were hitherto not stable enough, i.e. it could not be ensured that on withdrawal of A-beta oligomers from a preparation at different times the same A-beta aggregate species were always present.

The previously known oligomer preparations as a rule consist of various intermediate forms which are not of uniform size and hence are insufficiently reproducible.

However, a range of compounds have been described which bind to anti-amyloid monoclonal antibodies. Such compounds are for example known from Manea et al., (Biopolymeres Peptide Science 2004, Vol. 76, p. 503-511 and Peptid-Science 2008, Vol. 90, No. 2, p. 94-104) and Chafekar et al. (ChemBioChem 2007, 8, 1857-1864). These are based on polymers to which A-beta epitopes (4-10) or (16-20) are bound. A method for detecting beta-amyloid peptides, wherein antibodies which bind to positions 13-28 and 1-16 of the peptide are used is known from U.S. Pat. No. 5,593,846. However, here not exclusively Aβ oligomers, but also monomers, are detected.

Particularly in the field of detection of A-beta oligomers in samples from tissue or body fluids, A-beta oligomers which were prepared from synthetically produced A-beta monomers using various protocols were hitherto used as the comparison value. It was not ensured that only one oligomer size was present in the oligomer preparations, and the preparations contained no A-beta monomers or fibrils.

Further, these samples did not display adequate storage stability and changed their properties as regards the oligomer-monomer composition.

Because of these disadvantages, exact calibration and characterization of oligomer detection systems was previously imprecise or rather impossible. In particular, worldwide harmonization and hence establishment of test systems was rendered difficult by the different protocols in the various laboratories.

There is thus a need for standards for quantifying pathogenic aggregates or oligomers of endogenous proteins which cause and/or characterize a protein misfolding disease, amyloid degeneration or protein aggregation disease.

The purpose of the present invention was to provide standards which render an exact and quantitative determination of pathogenic aggregates or oligomers of endogenous proteins possible.

The standards should be usable as internal or external standards.

A further purpose of the present invention was to provide homogeneous and stable preparations of standards for quantifying pathogenic aggregates or oligomers of endogenous proteins.

This problem is solved by standards for quantifying oligomers or pathogenic aggregates which characterize a protein aggregation disease or amyloid degeneration or protein misfolding disease, characterized in that a polymer is constructed from polypeptide sequences which with regard to their sequence are identical in the corresponding sub-segment with the endogenous proteins or exhibit a homology of at least 50% over the corresponding sub-segment with the endogenous proteins which characterize a protein aggregation disease or amyloid degeneration or protein misfolding disease, wherein the polymers do not aggregate.

In the sense of the present invention, standard describes a generally valid and accepted, fixed reference quantity which is used for comparison and determination of properties and/or quantity, in particular for determining the size and quantity of pathogenic aggregates of endogenous proteins. The standard in the sense of the present invention can be used for the calibration of instruments and/or measurements.

In the sense of the present invention, amyloid degenerations and protein misfolding diseases can also be combined under the term “protein aggregation disease”. Examples of such diseases and the endogenous proteins associated therewith are: A-beta and tau protein for AD, alpha synuclein for Parkinson's or prion protein for prion diseases, for example such as human Creutzfeld-Jakob disease (CJD), the sheep disease scrapie and bovine spongiform encephalopathy (BSE).

In the sense of the invention “homologous sequences” means that an amino acid sequence exhibits an identity with an amino acid sequence from an endogenous pathogenic aggregate or oligomers, which causes a protein aggregation disease, of at least 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%. In the present description, instead of the term “identity”, the terms “homologous” or “homology” are used synonymously. The identity between two nucleic acid sequences or polypeptide sequences is calculated by comparison by means of the program BESTFIT based on the algorithm of Smith, T. F. and Waterman, M. S (Adv. Appl. Math. 2: 482-489 (1981)) with setting of the following parameters for amino acids: gap creation penalty: 8 and gap extension penalty: 2; and the following parameters for nucleic acids: gap creation penalty: 50 and gap extension penalty: 3. Preferably the identity between two nucleic acid sequences or polypeptide sequences is defined by the identity of the nucleic acid sequence/polypeptide sequence over the whole respective sequence length, as calculated by comparison by means of the program GAP based on the algorithm of Needleman, S. B. and Wunsch, C. D. (J. Mol. Biol. 48: 443-453) with setting of the following parameters for amino acids: gap creation penalty: 8 and gap extension penalty: 2; and the following parameters for nucleic acids gap creation penalty: 50 and gap extension penalty: 3.

Two amino acid sequences are identical in the sense of the present invention if they possess the same amino acid sequence.

The term “corresponding sub-segment” of endogenous proteins should be understood to mean that peptide sequence which according to the definitions according to the invention exhibits an identical or with the stated percentage homologous peptide sequence of a monomer from which the standards according to the invention are constructed.

It is essential for the standards according to the invention that the standards do not aggregate, preferably due to the use of monomeric sequences which do not aggregate, since the “corresponding sub-segment” of endogenous proteins is not responsible for the aggregation, or the groups responsible for the aggregation do not aggregate because of blocking.

Aggregates in the sense of the present invention are

particles which consist of several, preferably identical building blocks which are not bound covalently to one another and/or

non-covalent agglomerations of several monomers.

In one implementation of the present invention, the standards have a precisely defined number of epitopes which are covalently linked to one another (directly or via amino acids, spacers and/or functional groups) for the binding of the relevant probes. Probes in the sense of the invention are selected from the group consisting of: antibodies, nanobody and affibody. Furthermore, probes are all molecules which possess adequate binding specificity for the aggregate to be detected, e.g. dyes (thioflavin T, Congo red, etc.).

The number of epitopes is determined by using a polypeptide sequence which with regard to its sequence is identical with that sub-segment of the endogenous proteins which forms an epitope or exhibits homology of at least 50% with this sub-segment, and also possesses the biological activity of the epitope. A polypeptide sequence thus selected is incorporated in the desired number during the construction of the standards according to the invention and/or linked together according to the invention.

The standards according to the invention are polymers which are made up of the above-described polypeptide sequences, preferably epitopes, optionally containing further components.

In a further implementation of the present invention, the above-described polypeptide sequences, preferably epitopes, and/or homologs thereof with the biological activity of the corresponding epitope, represent the equal or greatest number of monomers based on the number in each case of one of the residual monomer species of the standard and/or based on the number of all other monomers.

In a further implementation of the present invention, the epitopes are epitopes of the A-beta peptide selected from the sub-segments A-beta 1-8 (SEQ ID No. 2), A-beta 1-11 (SEQ ID No. 3), A-beta 1-16 (SEQ ID No. 4), A-beta 3-11 (SEQ ID No. 5) and pyroGluA-beta 3-11 (SEQ ID No. 6), A-beta 11-16 (SEQ ID No. 7) and pyroGluA-beta 11-16 (SEQ ID No. 8), for example of the human N-terminal epitope (with the following sequence: DAEFRHDSGYE (1-11; corresponds to SEQ ID No. 3).

PyroGlu is the abbreviation for a pyroglutamate which can be formed at position 3 and/or 11 of the A-beta peptide, after the residues lying N-terminal therefrom have been removed.

The standard molecule according to the invention is a polymer of the polypeptide sequences defined above. Under oligomer in the sense of the invention, a polymer is formed from 2, 3, 4, 5, 6, 7, 8, 9, 10, 11, 12, 13, 14, 15, 16, 17, 18, 19 or 20 monomers (monomer should be understood to mean the aforesaid polypeptide sequence), or multiples thereof, preferably 2-16, 4-16, 8-16, particularly preferably 8 or 16, or multiples thereof.

The standards according to the invention are thus oligomers or polymers according to the invention.

In one alternative of the present invention, the standards are water-soluble.

In one alternative of the present invention, the standards according to the invention are made up of identical polypeptide sequences.

In one alternative of the present invention, the standards according to the invention are made up of different polypeptide sequences.

In one alternative of the present invention, such above-defined polypeptide sequences are concatenated in a linear conformation.

In one alternative of the present invention, such above-defined polypeptide sequences are concatenated in a branched oligomer according to the invention.

In one alternative of the present invention, such above-defined polypeptide sequences are concatenated in a cross-linked oligomer according to the invention.

Branched or cross-linked oligomers according to the invention can be produced by linking individual building blocks by means of lysine or by means of click chemistry.

As described above, the standards according to the invention, that is the oligomers or polymers according to the invention, in addition to the polypeptide sequences, preferably epitopes, present in precisely defined number, can further contain additional amino acids, spacers and/or functional groups, via which the polypeptide sequences, preferably epitopes, are covalently linked to one another.

In one alternative, the direct linkage of the polypeptide sequences, preferably epitopes with cysteine, in particular by disulfide bridging by cysteines is excluded (in order to avoid reducing agents removing the bridging). Likewise in a further modification, direct linkage of the spacers with the polypeptide sequence on the one hand and with cysteine on the other is excluded.

In one alternative, the invention relates to a standard molecule, containing or made up of copies of the amino-terminal part of the A-beta peptide, selected from the sub-segments A-beta 1-8 (SEQ ID No. 2), A-beta 1-11 (SEQ ID No. 3), A-beta 1-16 (SEQ ID No. 4), A-beta 3-11 (SEQ ID No. 5) and pyroGluA-beta 3-11 (SEQ ID No. 6), A-beta 11-16 (SEQ ID No. 7) and pyroGluA-beta 11-16 (SEQ ID No. 8), for example of the human N-terminal epitope (with the following sequence: DAEFRHDSGYE (1-11).

The duplication of the epitopes via functional groups can be performed before or after the synthesis of the individual building blocks. The covalent linkage of the polypeptide sequences is characteristic for the standards according to the invention.

The polypeptide sequences to be used according to the invention can be identical with the sequence of the A-beta full-length peptide or exhibit a homology of 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% with the sequence of the A-beta full-length peptide.

Alternatively, polypeptide sequences which are identical with a sub-segment of the A-beta full-length peptide, or exhibit a homology of 50, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% with a sub-segment of the A-beta full-length peptide, are also used for constructing the standard molecules according to the invention.

Essential for the sequences used according to the invention is their property of not aggregating (or only in a controlled manner depending on the conditions) and/or their the activity as epitope.

In a further implementation of the present invention, the standards are constructed as dendrimers. The dendrimers according to the invention are constructed of the above-described polypeptide sequences to be used according to the invention and can contain a central scaffold molecule. Preferably the scaffold molecule is a streptavidin monomer, particularly preferably a polymer, in particular tetramer.

In one modification, the dendrimers according to the invention contain polypeptide sequences which possess a sequence which is identical with a sub-segment of the A-beta peptide, or exhibits at least 50% homology to the corresponding sub-segment.

According to the invention, the term at least 50% homology should also be understood to mean a higher homology selected from the group consisting of 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

In one implementation of the invention, standards, advantageously with higher solubility in the aqueous than pathogenic aggregates or oligomers of endogenous proteins, are formed of polypeptide sequences which are identical with the N-terminal region of the A-beta peptide or exhibit at least 50% homology thereto. According to the invention, the N-terminal region of an A-beta polypeptide should be understood to mean the amino acid sequence A-beta 1-8 (SEQ ID No. 2), A-beta 1-11 (SEQ ID No. 3), A-beta 1-16 (SEQ ID No. 4), A-beta 3-11 (SEQ ID No. 5) and pyroGluA-beta 3-11 (SEQ ID No. 6), A-beta 11-16 (SEQ ID No. 7) and pyroGluA-beta 11-16 (SEQ ID No. 8).

A standard molecule according to the invention can contain epitopes for at least 2, 3, 4, 5, 6, 7, 8, 9, 10 or more different probes.

Epitopes characteristic for different probes can be incorporated into the standards according to the invention by using polypeptide sequences which are identical with different regions of the A-beta peptide or exhibit at least 50% homology thereto, but possess the activity of the corresponding epitope.

In one implementation, polypeptide sequences which are identical or exhibit 50% homology with the N-terminal region of the A-beta polypeptide and polypeptide sequences which are identical or exhibit at least 50% homology with the C-terminus of the A-beta polypeptide are used for this.

In one implementation of the present invention, the standard molecules contain so-called spacers.

A spacer should be understood to mean a molecule which is incorporated into the standard molecule via covalent bonds, and possesses defined physical and/or chemical properties, through which the properties of the standard molecules are modified. In one implementation of the standards according to the invention, hydrophilic or hydrophobic, preferably hydrophilic spacers are used. Hydrophilic spacers are selected from the group of molecules made up of polyethylene glycol, sugars, glycerin, poly-L-lysine or beta-alanine.

In one alternative of the present invention, the standards according to the invention contain (further) functional groups.

Functional groups should be understood to mean molecules which are covalently bound to the standard molecules. In one modification, the functional groups contain biotin groups. As a result, strong covalent bonding to streptavidin is enabled. Standard molecules containing biotin groups can thus be bound to molecules containing streptavidin groups. If the standard molecules according to the invention contain biotin and/or streptavidin groups, larger standards can thus be assembled or several optionally different standard molecules, be bound onto one scaffold.

In a further alternative of the present invention, the standard molecules contain dyes for spectrophotometric determination and/or aromatic amino acids. Aromatic amino acids are e.g. tryptophans, tyrosine, phenylalanine or histidine, or selected from this group. Through the incorporation of tryptophan, spectrophotometric determination of the concentration of standards in solution is enabled.

A further subject of the present invention are dendrimers containing polypeptides which with regard to their sequence are identical in the corresponding sub-segment with the endogenous proteins or exhibit a homology of at least 50% over the corresponding sub-segment with the endogenous proteins which characterize a protein aggregation disease.

The dendrimers according to the invention can contain any of the above-described features of the standards or any desired combination thereof.

In one alternative of the present invention, these are:

dendrimers containing a precisely defined number of epitopes for the covalent binding of probes,

dendrimer containing epitopes of the A-beta peptide,

dendrimer characterized in that it possesses a higher solubility in the aqueous than the pathogenic aggregates of endogenous proteins which characterize a protein aggregation disease,

dendrimer containing functional groups,

dendrimer containing at least one spacer molecule and/or

dendrimer containing dyes for spectrophotometric determination and/or aromatic amino acids.

According to the invention, the dendrimers have radial symmetry.

In one modification, the branching of the first generation of the dendrimer is effected via lysine, in particular three lysine amino acids.

In a further alternative of the present invention, in the standards, in particular dendrimers, the polypeptide sequences, preferably epitopes, are linked, in particular covalently bound to one another or to other components of the standard such as amino acids, spacers and/or functional groups and/or other above-described components, not via a bond to a sulfur atom, not via a thioether bond and/or not via cysteine (optionally by disulfide bridging via cysteine). Likewise in a further modification, the polypeptide sequences, preferably epitopes, and a spacer bound thereto on the spacer are linked, in particular covalently bound to one another or to other components of the standard such as amino acids, further spacers and/or functional groups and/or other above-described components not via a bond to a sulfur atom, not via a thioether bond and/or not via cysteine.

The present invention further relates to a method for the production of a standard, as described above.

In one implementation, the standard according to the invention is produced by peptide synthesis or recombinant methods which are known to those skilled in the art.

A further subject of the present invention is use of an above-described standard or an above-described dendrimer for quantifying pathogenic aggregates or oligomers of endogenous proteins which characterize a protein aggregation disease.

In one implementation of the invention, the standard is used to quantify A-beta oligomers.

Hence a method for quantifying pathogenic aggregates or oligomers of endogenous proteins which characterize a protein aggregation disease or amyloid degeneration or protein misfolding disease, wherein the oligomers or polymers according to the invention are used as a standard is also a subject of the present invention.

The standards according to the invention are used in one implementation of the present invention for calibration in the surface FIDA method, Elisa (sandwich Elisa) or FACS.

In another embodiment, the present invention relates to a kit which comprises standard according to the invention. The compounds and/or components of the kit of the present invention can be packed in containers optionally with/in buffers and/or solution. Alternatively, a number of components can be packed in the same container. In addition to this or alternatively to this, one or more of the components could be absorbed on a solid support, such as for example a glass plate, a chip or a nylon membrane or on the well of a microtiter plate. Further, the kit can contain directions for the use of the kit for any one of the embodiments.

In one alternative of the present invention, the standards for quantifying pathogenic aggregates or oligomers of endogenous proteins are used in that:

in a first step, the standards or the dendrimers are marked with probes and the number of the probe bound to the standards or dendrimers is determined,

in a second step, pathogenic aggregates or oligomers of endogenous proteins which characterize a protein aggregation disease are marked with probes, the number of the probes binding respectively to a pathogenic aggregate or oligomer is determined,

in a third step, the number of probes binding respectively to a standard or dendrimer from step 1 is compared with that from step 2, and

in a fourth step, the number and the size of the oligomers from the body fluid is thereby determined.

In one modification of the present invention, the standards according to the invention, preferably dendrimers, are used for the calibration of the surface FIDA method. In a first step, endogenous pathogenic aggregates from body fluids, e.g. A-beta aggregates, are immobilized on a glass surface by a capture molecule, e.g. capture antibody. In the case of A-beta aggregates an N-terminally binding capture antibody can be used for this. After the immobilization, the aggregates are marked by two different probes. In the case of A-beta aggregates, A-beta antibodies which are both bound via an N-terminal binding epitope are for example used. The detection probes are marked with preferably different fluorescent dyes. They thereby become visible under the microscope, e.g. laser scanning microscope.

According to the invention, monomer detection of endogenous polypeptides is excluded since in the test system three different or three differently marked probes which bind to a similar or identical epitope are used. Alternatively or in addition, the detection of monomers can be excluded in that signals with a lower intensity are not assessed because of the definition of an intensity threshold value. Since larger aggregates possess several binding sites for the two probes with different marked dyes, monomer detection can alternatively or additionally be excluded by cross-correlation of these signals.

The standards according to the invention can be used as internal or external standards in the measurement.

### EXAMPLES

### 1. Preparation of Aβ Oligomer Standard

In a practical example, an Aβ oligomer standard was constructed which exhibited 16 epitopes for N-terminal-binding Aβ antibodies (epitope corresponds to Aβ₁₋₁₁, sequence: DAEFRHDSGYE, SEQ ID No. 3).

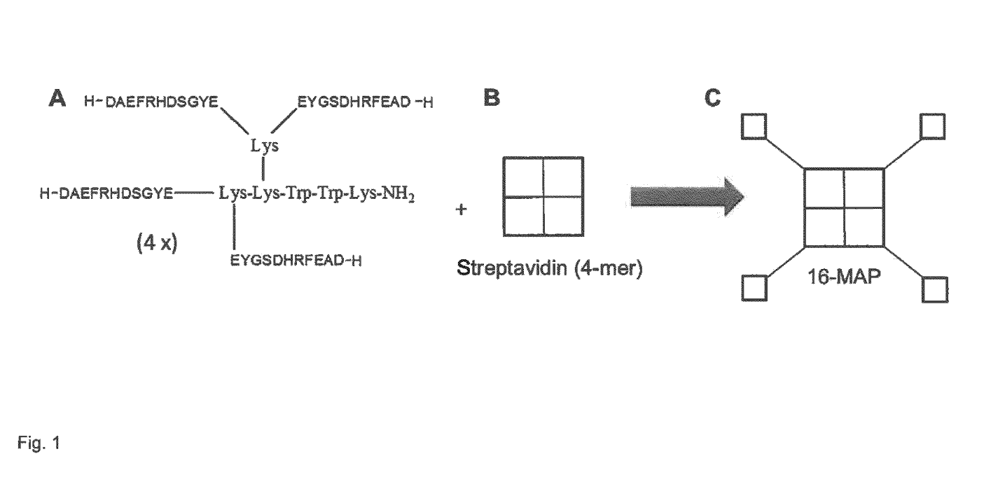

Firstly, a multiple antigen peptide (MAP) was synthesized which consisted of four N-terminal Aβ epitopes Aβ1-11. These were coupled in accordance with FIG. 1A to a triple lysine core, which for the precise determination of the MAP concentration by UV/VIS spectroscopy contained two tryptophans. In addition, a biotin tag was attached N-terminally. This was used for the coupling of respectively four 4-MAP units to each streptavidin tetramer, shown under B in FIG. 1. After incubation of 4-MAP and streptavidin, 16-MAP was formed, as shown under C in FIG. 1. 16-MAP was separated from other components of the incubation mixture by size exclusion chromatography.

Next, MAP-16 was serially diluted in PBS and used in the sFIDA test for the detection of Aβ oligomers.

### 2. Detection of Aβ Oligomers

a. Glass Plate Preparation

Glass microtiter plates were cleaned in an ultrasonic bath for 15 minutes and then treated with a plasma cleaner for 10 mins. For the activation of the glass surface, the wells were incubated in 5M NaOH for at least 3 hours, rinsed with water and then dried in the current of nitrogen gas. For the coating with dextran, the glass surface was hydroxylated and then activated with amino groups. For this, the glass plates was incubated overnight in a solution of 5M ethanolamine in DMSO. Next, the glass plates were rinsed with water and dried in a current of nitrogen gas. Carboxymethyl dextran (CMD) was dissolved in water at a concentration of 20 mg per ml and mixed with N-ethyl-N-(3-dimethylaminopropyl) carbodiimide (EDC), (200 mM) and N-hydroxysuccinimide (NHS), (50 mM). After a preincubation of 10 minutes, the solution was incubated for a further 2 hours at room temperature. Then the glass plates were washed with water.

b. Immobilization of Antibodies as Capture Molecules on the Coated Glass

A second activation was effected with a solution of EDC/NHS (200 or 50 mM) for 5 minutes. The solution of the antibodies was added to this and incubated for 2 hours at 4° C. As a result, the antibodies were covalently bound onto the CMD-activated glass surface. In order then to deactivate remaining active carboxyl terminal groups on the CMD spacer, this was incubated for 5 minutes with 1M ethanolamine in DMSO. The glass was then washed three times with PBS.

c. Immobilization of MAP-16 on the Pretreated Glass

The MAP-16-containing sample to be assayed was incubated for 1 hour on the glass, then washed three times with TBST (0.1%) (W/W), Tween-20 in TBS buffer, TBS: 50 nM Tris-HCl, 0.15 M NaCl, pH 7.4).

d. Labeling of the Probes with Fluorescent Dye

6E10 Alexa-488 antibodies and IC-16 antibodies were used. The IC16 antibodies were marked with a kit (Fluorescence labeling KIT Alexa-647, Molecular Probes, Karlsruhe, Germany) according to the manufacturer's instructions. The labeled antibodies were stored in PBS containing 2 mM sodium azide at 4° C. in the dark.

e. Marking of the Aggregates with the Probes

The probes were added and incubated for 1 hour at room temperature, then washed five times with TBST and twice with water.

f. Detection of the Aggregate Standard

The measurement was effected with a confocal laser scanning microscope LSM 710 (Carl Zeiss, Jena, Germany). The microscope was equipped with an argon ion laser and three helium-neon lasers. The measurements were effected in tile scan mode, in which adjacent surfaces in a well are measured and assembled to an image. Each tile scan contained 3×2 individual images, and each image had an area of 213×213 μm.

Alternatively, the measurements were effected on a TIRF microscope (TIRF=total internal reflection) consisting of an inverted microscope DMI 6000, a laser box and a Hamamatsu EM-CCD C9100 camera. In the tile scan mode 3×3 individual images each with a size of 109.9×109.9 μm were.

The assessment was effected with the software “Image J” ((http://rsbweb.nih.gov/ij/). Through the use of different probes, a colocalization analysis could be performed. For this, firstly a cut-off value, defined by a negative control without MAP-16, was subtracted from the intensity values of the individual pixels. Next, the number of colocalized pixels whose intensity was greater than zero was added.

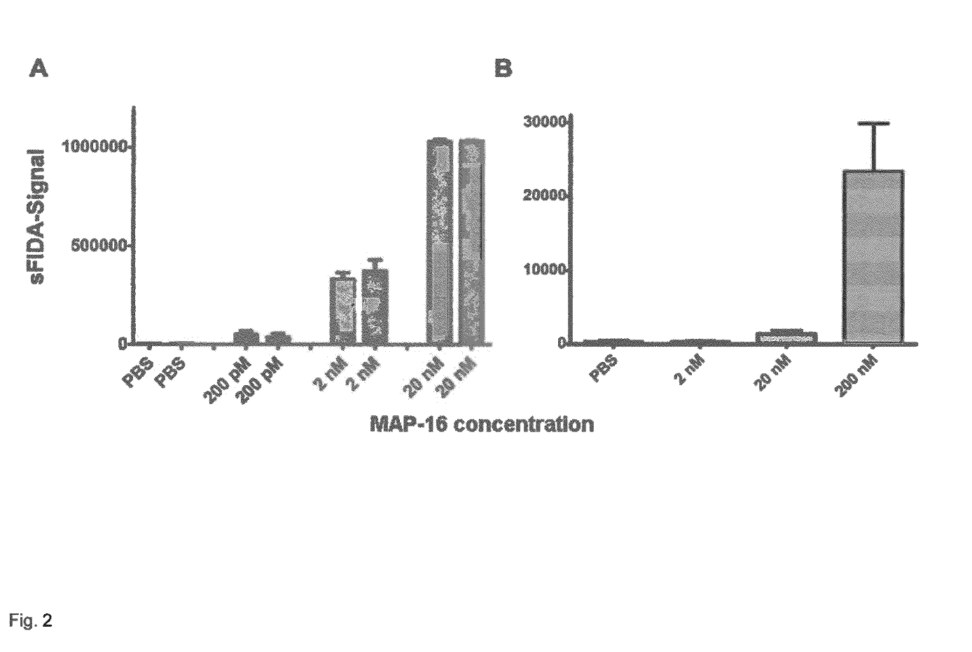

FIG. 2 shows the results of the measurements. It can clearly be discerned that the sFIDA signal, i.e. the quantity of the colocalized pixels, correlates with the concentration of the MAP-16 molecules.

### DESCRIPTION OF DIAGRAMS

FIG. 1: Construction of an Aβ oligomer standard with 16 epitopes for N-terminal-binding Aβ antibodies which correspond to the first 11 amino acids of Aβ (sequence: DAEFRHDSGYE). A) 4-MAP was synthesized, consisting of 4 N-terminal Aβ epitopes 1-11 coupled to a threefold lysine core which contained two tryptophans for the concentration determination by UV/VIS spectroscopy. B and C) For the production of 16-MAP in each case four 4-MAP were coupled via a streptavidin teramer. MAP-16 was separated from other components of the incubation mixture by means of size exclusion chromatography.

FIG. 2: sFIDA measurements of MAP-16 at various concentrations, diluted in PBS buffer. PBS buffer with no MAP-16 was used as the negative control. A) The measurements were performed on a laser scanning microscope (Zeiss LSM 710). B). The measurements were performed on a TIRF microscope (Leica).

1.-19. (canceled)

20. A standard for quantifying pathogenic aggregates or oligomers of endogenous proteins which characterize a protein aggregation disease or amyloid degeneration or protein misfolding disease, wherein the standard comprises non-aggregating polymers constructed from polypeptide sequences which with respect to their sequence are identical in a sub-segment with the endogenous proteins or exhibit homology of at least 50% across the sub-segment with the endogenous proteins.

21. The standard of claim 20, wherein the standard comprises a precisely defined number of epitopes for binding probes, which epitopes are covalently linked together.

22. The standard of claim 20, wherein the standard comprises epitopes of A-beta peptide.

23. The standard of claim 22, wherein the standard comprises at least one epitope selected from A-beta 1-8 (SEQ ID No. 2), A-beta 1-11 (SEQ ID No. 3), A-beta 1-16 (SEQ ID No. 4), A-beta 3-11 (SEQ ID No. 5), pyroGluA-beta 3-11 (SEQ ID No. 6), A-beta 11-16 (SEQ ID No. 7), pyroGluA-beta 11-16 (SEQ ID No. 8).

24. The standard of claim 20, wherein the standard is soluble in an aqueous medium.

25. The standard of claim 20, wherein the standard comprises functional groups.

26. The standard of claim 20, wherein the standard comprises at least one spacer molecule.

27. The standard of claim 20, wherein the standard comprises at least one of a dye suitable for spectrophotometric determination and an aromatic amino acid.

28. The standard of claim 20, wherein the polypeptide sequences are not linked to one another or to other components of the standard via a bond to a sulfur atom, via a thioether bond and/or via cysteine.

29. The standard of claim 20, wherein the polypeptide sequences are bound to one another in a linear, branched or cross-linked conformation, or are present as dendrimer.

30. A dendrimer, wherein the dendrimer comprises polypeptides which with respect to their sequence are identical in a sub-segment or exhibit homology of at least 50% across the sub-segment with endogenous proteins which characterize a protein aggregation disease, and wherein polymers comprising the polypeptides do not aggregate.

31. A method for the production of the standard of claim 20, wherein the method comprises a peptide synthesis or a recombinant method.

32. A method of quantifying pathogenic aggregates or oligomers of endogenous proteins which characterize a protein aggregation disease, wherein the method comprises employing the standard of claim 20.

33. The method of claim 32, wherein A-beta oligomers are quantified.

34. The method of claim 32, wherein at least one of surface FIDA method, Elisa, sandwich Elisa or FACS is calibrated.

35. The method of claim 32, wherein the method comprises:
marking the standard with probes and determining a number of probes bound to the standard,
marking pathogenic aggregates or oligomers of endogenous proteins which characterize a protein aggregation disease with probes and determining a number of probes which bind to a pathogenic aggregate or oligomer,
comparing the determined number of probes binding to the standard with the determined number of probes which bind to a pathogenic aggregate or oligomer, and
determining number and size of the oligomers.

36. A kit for quantifying pathogenic aggregates or oligomers of endogenous proteins which characterize a protein aggregation disease, wherein the kit comprises the standard of claim 20.

37. A method of quantifying pathogenic aggregates or oligomers of endogenous proteins which characterize a protein aggregation disease, wherein the method comprises employing the dendrimer of claim 30.

38. The method of claim 37, wherein the method comprises:
marking the dendrimer with probes and determining a number of probes bound to the dendrimer,
marking pathogenic aggregates or oligomers of endogenous proteins which characterize a protein aggregation disease with probes and determining a number of probes which bind to a pathogenic aggregate or oligomer,
comparing the number of probes binding to the dendrimer with the number of probes which bind to a pathogenic aggregate or oligomer, and
determining number and size of the oligomers.

39. A kit for quantifying pathogenic aggregates or oligomers of endogenous proteins which characterize a protein aggregation disease, wherein the kit comprises the dendrimer of claim 30.


SEQUENCE LISTING (8 sequences)

SEQ ID NO: 1 - protein, 43 aa, Homo sapiens
DAEFRHDSGYEVHHQKLVFFAEDVGSNKGAIIGLMVGGVVIAT

SEQ ID NO: 2 - protein, 8 aa, Artificial Sequence
  Description of Artificial Sequence Synthetic peptide
DAEFRHDS

SEQ ID NO: 3 - protein, 11 aa, Artificial Sequence
  Description of Artificial Sequence Synthetic peptide
DAEFRHDSGYE

SEQ ID NO: 4 - protein, 16 aa, Artificial Sequence
  Description of Artificial Sequence Synthetic peptide
DAEFRHDSGYEVHHQK

SEQ ID NO: 5 - protein, 9 aa, Artificial Sequence
  Description of Artificial Sequence Synthetic peptide
EFRHDSGYE

SEQ ID NO: 6 - protein, 9 aa, Artificial Sequence
  Description of Artificial Sequence Synthetic peptide
EFRHDSGYE

SEQ ID NO: 7 - protein, 6 aa, Artificial Sequence
  Description of Artificial Sequence Synthetic peptide
EVHHQK

SEQ ID NO: 8 - protein, 6 aa, Artificial Sequence
  Description of Artificial Sequence Synthetic peptide
EVHHQK